(19) United States
(12) Patent Application Publication
(10) Pub. No.: US 2016/0213506 A1
(43) Pub. Date: Jul. 28, 2016

(54) REHABILITATION FOOTWEAR

(71) Applicant: Tung-Cheng CHEN, Taichung City (TW)

(72) Inventor: Tung-Cheng CHEN, Taichung City (TW)

(21) Appl. No.: 14/604,844

(22) Filed: Jan. 26, 2015

(51) Int. Cl. A61F 5/01; A61F 5/30

(52) U.S. Cl. CPC A61F 5/0195; A61F 5/0111; A61F 5/30

(57) ABSTRACT

A rehabilitation footwear has a sole, two supporting arms and multiple fastening belts. The sole has two sidewalls formed on and protrude from a top of the sole and facing to each other. The supporting arms are respectively connected to the sidewalls of the sole and each has an inserting tab, multiple supporting tabs, at least two bending segments and multiple belt mounts. The inserting tab is connected to one of the sidewalls of the sole. The supporting tabs are sequentially formed with each other. The at least two bending segments are formed with the supporting tabs, and each bending segment is formed with two adjacent supporting tabs and has a convex inner side and a concave outer side. The belt mounts are respectively mounted on the supporting mounts. The fastening belts are connected to the sole and the supporting arms.

### BACKGROUND OF THE INVENTION

1. Field of the Invention

The present invention relates to a rehabilitation footwear, and more particularly to a rehabilitation footwear that can be adjusted to fit with the lower leg of a patient to provide a preferred rehabilitation effect to the patient.

2. Description of Related Art

With reference to FIG. 6, a conventional rehabilitation footwear 50 is used on a lower leg of a patient and has a sole 51, two supporting arms 52 and multiple fastening belts 53. The sole 51 has a bottom and two sidewalls 511. The sidewalls 511 of the sole 51 are formed on and protrude from the bottom of the sole 51 and face to each other. The supporting arms 52 are made of plastic and are respectively connected to the sidewalls 511 of the sole 51 to support the lower leg of the patient. Each one of the supporting arms 52 has a flat inner side 521 and a flat outer side 522. The inner sides 521 of the supporting arms 52 face to each other. The fastening belts 53 are connected to the sole 51 and the supporting arms 52 to hold the patient's lower leg and foot with the conventional rehabilitation footwear 50.

However, the conventional rehabilitation footwear 52 can provide a protecting and rehabilitating effect to the patient's injured lower leg and foot. When conventional rehabilitation footwear 50 is used to the patients with different sizes of lower legs, the inner sides 521 and the outer sides 522 of the supporting arms 52 of the conventional rehabilitation footwear 50 are flat and cannot be bent relative to the patients' lower legs to fully abut the patients' lower legs. Then, the supporting arms 52 have gaps relative to the patient's lower leg and cannot fit with the different sizes of the lower legs and this will decrease the rehabilitation effect to the patients.

The present invention provides a rehabilitation footwear to mitigate or obviate the aforementioned problems.

### SUMMARY OF THE INVENTION

The main objective of the present invention is to provide a rehabilitation footwear that can be adjusted to fit with the lower leg of a patient to provide a preferred rehabilitation effect to the patient.

The rehabilitation footwear in accordance with the present invention has a sole, two supporting arms and multiple fastening belts. The sole has two sidewalls formed on and protrude from a top of the sole and facing to each other. The supporting arms are respectively connected to the sidewalls of the sole and each has an inserting tab, multiple supporting tabs, at least two bending segments and multiple belt mounts. The inserting tab is connected to one of the sidewalls of the sole. The supporting tabs are sequentially formed with each other. The at least two bending segments are formed with the supporting tabs, and each bending segment is formed with two adjacent supporting tabs and has a convex inner side and a concave outer side. The belt mounts are respectively mounted on the supporting mounts. The fastening belts are connected to the sole and the supporting arms.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a rehabilitation footwear in accordance with the present invention;

FIG. 2 is an exploded perspective top view of the rehabilitation footwear in FIG. 1;

FIG. 3 is an enlarged exploded perspective bottom view of the rehabilitation footwear in FIG. 2;

FIG. 4 is an operational front side view of the rehabilitation footwear in FIG. 1;

FIG. 5 is another operational front side view of the rehabilitation footwear in FIG. 1; and

FIG. 6 is an operational front side view of a rehabilitation footwear in accordance with the prior art.

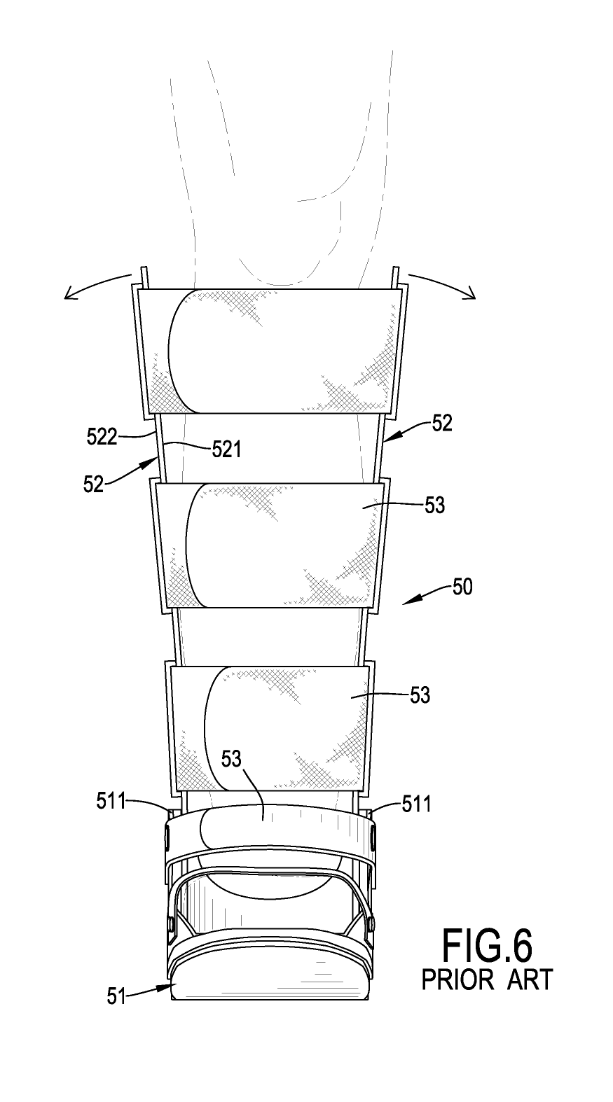

### DETAILED DESCRIPTION OF THE PREFERRED EMBODIMENT

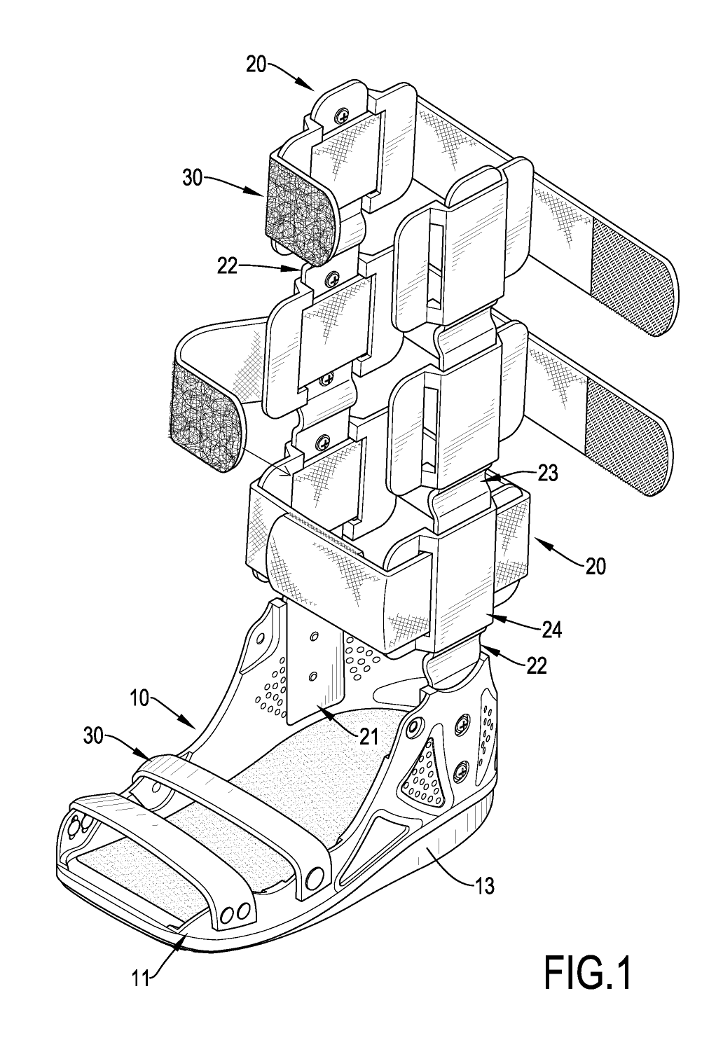

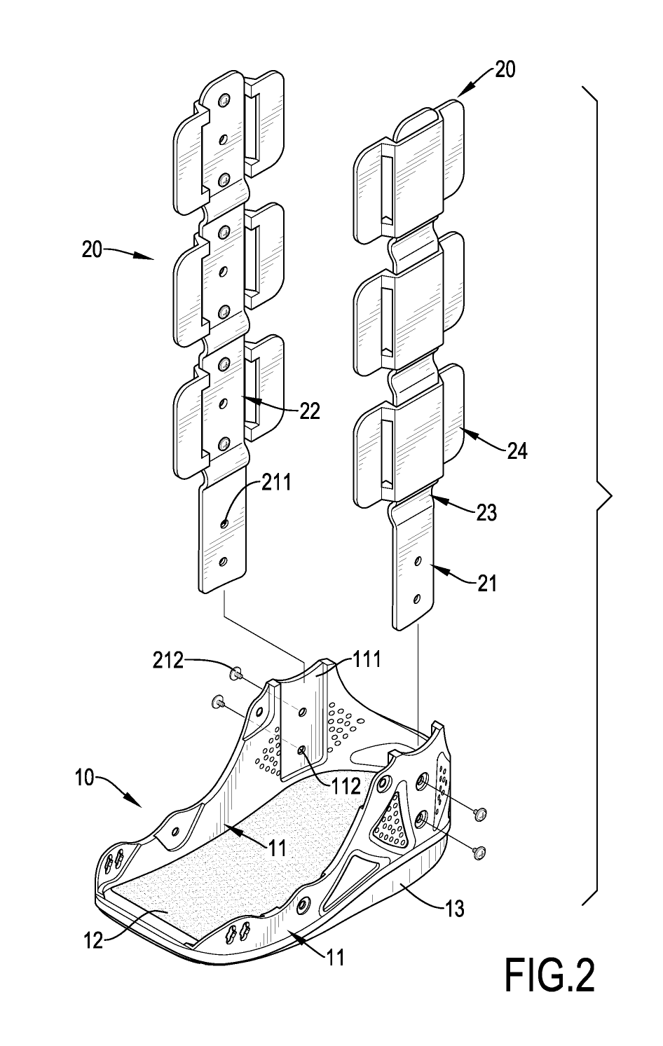

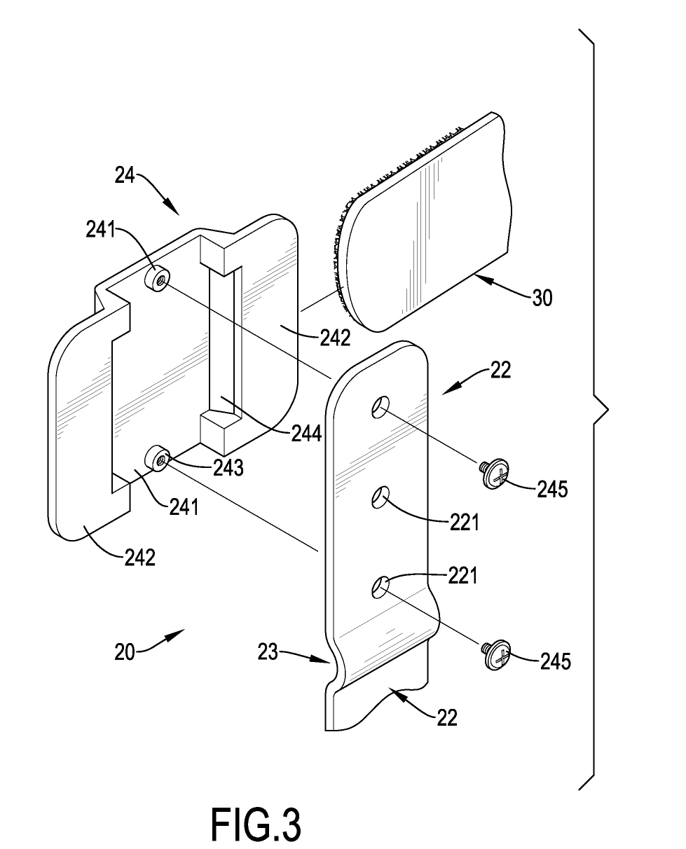

With reference to FIGS. 1 to 3, a rehabilitation footwear in accordance with the present invention has a sole 10, two supporting arms 20 and multiple fastening belts 30.

The sole 10 has a rear end, a front end, a top, a bottom, two sidewalls 11, a pad 12 and a shock-absorbing layer 13. The sidewalls 11 are formed on and protrude from the top of the sole 10 form the front end to the rear end of the sole 10 and face to each other. Preferably, the sidewalls 11 of the sole 10 are connected to each other at the rear end of the sole 10.

In addition, each one of the sidewalls 11 of the sole 10 has an inner side, a top edge, an inserting recess 111 and at least one through hole 112. The inner sides of the sidewalls 11 face to each other. The inserting recess 111 is formed in the inner side of the sidewall 11 of the sole and is formed through the top edge of the sidewall 11 near the rear end of the sole 10. The at least one through hole 112 is formed through the sidewall 11 of the sole 10 and communicates with the inserting recess 111 of the sidewall 11.

The pad 12 is mounted on the top of the sole 10 between the front end, the rear end and the sidewalls 11 of the sole 10. The shock-absorbing layer 13 is mounted on the bottom of the sole 10 to provide a shock-absorbing effect to a patient.

The supporting arms 20 may be made of aluminum are respectively connected to the sidewalls 11 of the sole 10 and face to each other. Each one of the supporting arms 20 has a lower end, an upper end, an inserting tab 21, multiple supporting tabs 22, at least two bending segments 23 and multiple belt mounts 24.

The inserting tab 21 is formed on the lower end of the supporting arm 20, is mounted in the inserting recess 111 of one of the sidewalls 11 of the sole 10 from the top edge of the corresponding sidewall 11 of the sole 10 and has a top end and at least one connecting hole 211. The at least one connecting hole 211 is formed through the inserting tab 21 and aligns with the at least one through hole 112 of the corresponding sidewall 11 of the sole 10. Furthermore, the inserting tabs 21 is securely connected to the corresponding sidewall 11 of the sole 10 by a fastener 212 mounting through the at least one through hole 112 of the corresponding sidewall 11 of the sole 10 and connecting to the at least one connecting hole 211 of the inserting tab 21. Preferably, each one of the sidewalls 11 of the sole 10 has two through holes 112 and each one of the inserting tabs 21 has two connecting holes 211 and two fasteners 212.

The supporting tabs 22 are formed on the upper end of the supporting arm 20, are sequentially formed with each other and a lowest one of the supporting tabs 22 is connected to the inserting tab 21. Each one of the supporting tabs 22 has an inner side, an outer side and at least two mounting holes 221. The inner sides of the supporting tabs 22 of the supporting arm 20 face the inner sides of the supporting tabs 22 of the other one of the supporting arms 20. The at least two mounting holes 221 are formed through the supporting tab 22. Preferably, each one of the supporting tabs 22 has three mounting holes 221.

The at least two bending segments 23 are formed with the supporting tabs 22, and each one of the at least two bending segments 23 is formed with two adjacent supporting tabs 22 and has a convex inner side 231 and a concave outer side 232. The convex inner side 231 of each one of the at least two bending segments 23 extends out of the inner sides of the two adjacent supporting tabs 22. The concave outer side of each one of the at least two bending segments 23 is formed in the bending segment 23 between the outer sides of the two adjacent supporting tabs 22. Then, an angle between the two adjacent supporting tabs 22 can be adjusted via the bending segment 23.

Additionally, each one of the supporting arms 20 has three bending segments 23, two of the bending segments 23 are formed between the three supporting tabs 22 and the other one of the bending segments 23 is formed between the top end of the inserting tab 21 and the lowest supporting tab 22.

The belt mounts 24 are respectively mounted on the supporting tabs 22 of the supporting arm 20, and each one of the belt mounts 24 has a central segment 241, two wing segments 242, two connecting tubes 243 and two belt holes 244. The central segment 241 of the belt mount 24 is U-shaped and is mounted around the outer side of the supporting tab 22 and has an inner side and two opposite sidewalls. The inner side of the central segment 241 abuts the outer side of the supporting tab 22. The opposite sidewalls are formed with the inner side of the central segment 241, face to each other beside the supporting tab 22 and each one of the opposite sidewalls has a free edge opposite to the central segment 241.

The wing segments 242 are respectively formed on the free edges of the opposite sidewalls of the central segment 241 and align with each other. The connecting tubes 243 are formed on and protrude from the inner side of the central segment 241 at an interval and are respectively mounted in two of the at least two mounting holes 221 of the supporting tab 22. Furthermore, the belt mount 24 is securely mounted on the supporting tab 22 by two screws 245 connecting to the mounting tubes 243 to hold the belt mount 24 securely with the supporting tab 22. The belt holes 244 are respectively formed through the opposite sidewalls of the central segment 241 between the wing segments 24 and communicate with each other.

The fastening belts 30 are connected to the sole 10 and the supporting arms 20 to form a space between the sole 10 and the supporting arms 20 to hold a lower leg of a patient. In addition, the rehabilitation footwear has five fastening belts 30, three of the fastening belts 30 are mounted through the belts holes 244 of the belt mounts 24 to connect the supporting arms 20 to each other, and the other two of the fastening belts 30 are connected to the sidewalls 11 of sole 10.

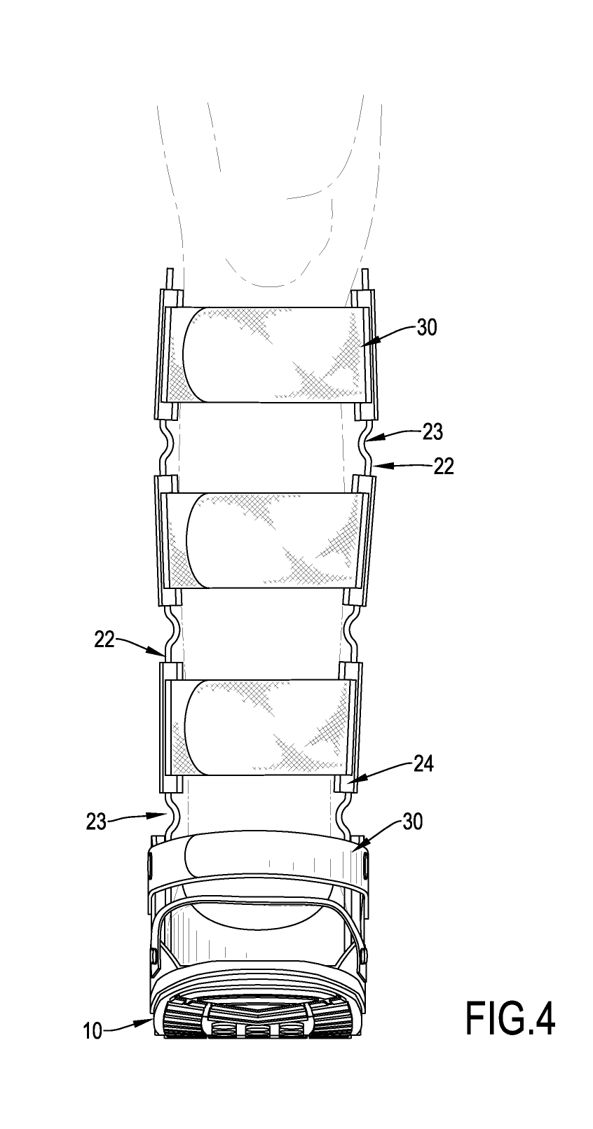

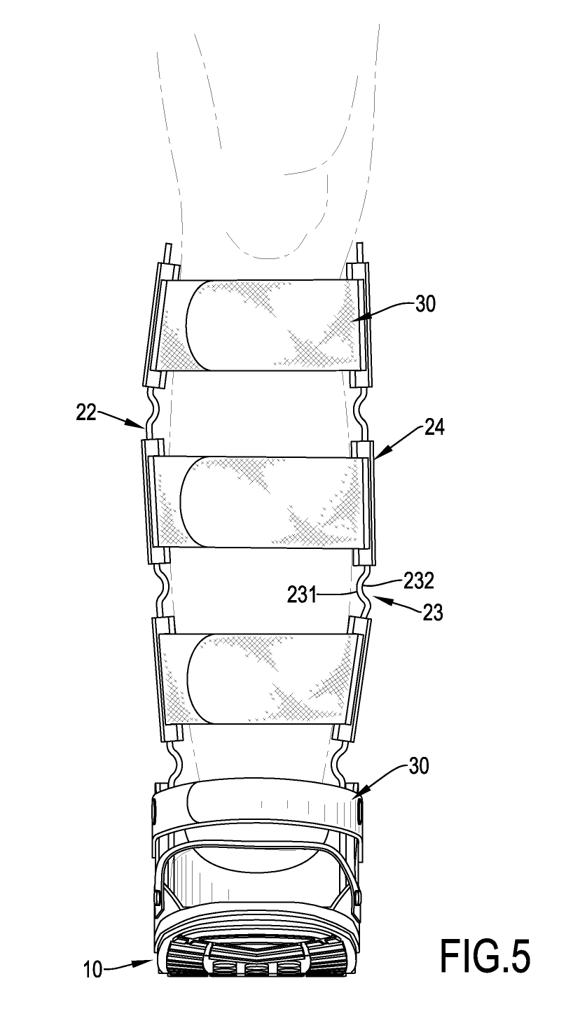

In use, with reference to FIGS. 4 and 5, when the rehabilitation footwear in accordance with the present invention is used to patients with different sizes of lower legs, the convex inner sides 231 and the concave outer sides 232 of the at least two bending segments 23 of the each one of the supporting arms 20 can be used to adjust an angle between two adjacent supporting tabs 22. Then, the supporting tabs 22 can be moved relative to each other according to the sizes of the patients' lower legs to abut the as far as possible to avoid gaps between the patient's lower leg and the supporting arms 20, and this can provide a preferred rehabilitation effect to the patients with different sizes of lower legs. Consequently, the patient's foot is mounted on the sole 10 and the patient's lower leg is held with the supporting arms 20 by the fastening belts 30. In addition, the belt mounts 24 can be separated from the supporting tabs 22 according to the user's need and the patient's lower leg is held with the supporting arms 20 by the fastening belts 30 directly to enable the rehabilitation footwear to meet the demand of patients with different sizes of lower legs.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A rehabilitation footwear comprising:
a sole having
a rear end;
a front end;
a top;
a bottom; and
two sidewalls formed on and protruding from the top of the sole form the front end to the rear end of the sole and facing to each other;
two supporting arms respectively connected to the sidewalls of the sole, facing to each other and each one of the supporting arms having
a lower end;
an upper end;
an inserting tab formed on the lower end of the supporting arm and connected to one of the sidewalls of the sole;
multiple supporting tabs formed on the upper end of the supporting arm, sequentially formed with each other and a lowest one of the supporting tabs connected to the inserting tab, and each one of the supporting tabs having an inner side and an outer side;
at least two bending segments formed with the supporting tabs, and each one of the at least two bending segments formed with two adjacent supporting tabs and having a convex inner side extending out of the inner sides of the two adjacent supporting tabs; and
a concave outer side formed in the bending segment between the outer sides of the two adjacent supporting tabs; and
at least one belt mount mounted on one of the supporting tabs of the supporting arm; and
multiple fastening belts connected to the sole and the supporting arms to form a space between the sole and the supporting arms.

2. The rehabilitation footwear as claimed in claim 1, wherein
each one of the supporting tabs has at least two mounting holes formed through the supporting tab; and
each one of the belt mounts has
a central segment being U-shaped and mounted around the outer side of a corresponding supporting tab and having
an inner side abuts the outer side of the corresponding supporting tab; and
two opposite sidewalls formed with the inner side of the central segment, facing to each other beside the corresponding supporting tab and each one of the opposite sidewalls having a free edge opposite to the central segment;
two wing segments respectively formed on the free edges of the opposite sidewalls of the central segment and aligning with each other;
two connecting tubes formed on and protruding from the inner side of the central segment at an interval and respectively mounted in two of the at least two mounting holes of the corresponding supporting tab; and
two belt holes respectively formed through the opposite sidewalls of the central segment between the wing segments and communicating with each other.

3. The rehabilitation footwear as claimed in claim 2, wherein the rehabilitation footwear has five fastening belts, three of the fastening belts are mounted through the belts holes of the belt mounts to connect the supporting arms to each other, and the other two of the fastening belts are connected to the sidewalls of sole.

4. The rehabilitation footwear as claimed in claim 3, wherein each one of the supporting arms has three supporting tabs and three bending segments, two of the bending segments are formed between the three supporting tabs and the other one of the bending segments is formed between the top end of the inserting tab and the lowest supporting tab.

5. The rehabilitation footwear as claimed in claim 4, wherein
each one of the sidewalls of the sole has
an inner side facing the inner side of the other one of the sidewalls of the sole;
a top edge;
an inserting recess formed in the inner side of the sidewall of the sole and is formed through the top edge of the sidewall near the rear end of the sole; and
at least one through hole formed through the sidewall of the sole and communicating with the inserting recess of the sidewall; and
the inserting tab of each one of the supporting arms is mounted in the inserting recess of one of the sidewalls of the sole from the top edge of a corresponding sidewall of the sole and having
a top end; and
at least one connecting hole formed through the inserting tab and aligning with the at least one through hole of the corresponding sidewall of the sole.

6. The rehabilitation footwear as claimed in claim 5, wherein the sole has
a pad mounted on the top of the sole between the front end, the rear end and the sidewalls of the sole; and
a shock-absorbing layer mounted on the bottom of the sole.

7. The rehabilitation footwear as claimed in claim 6, wherein the sidewalls of the sole are connected to each other at the rear end of the sole.

8. The rehabilitation footwear as claimed in claim 1, wherein each one of the supporting arms has three supporting tabs and three bending segments, two of the bending segments are formed between the three supporting tabs and the other one of the bending segments is formed between the top end of the inserting tab and the lowest supporting tab.

9. The rehabilitation footwear as claimed in claim 2, wherein each one of the supporting arms has three supporting tabs and three bending segments, two of the bending segments are formed between the three supporting tabs and the other one of the bending segments is formed between the top end of the inserting tab and the lowest supporting tab.

10. The rehabilitation footwear as claimed in claim 1, wherein
each one of the sidewalls of the sole has
an inner side facing the inner side of the other one of the sidewalls of the sole;
a top edge;
an inserting recess formed in the inner side of the sidewall of the sole and is formed through the top edge of the sidewall near the rear end of the sole; and
at least one through hole formed through the sidewall of the sole and communicating with the inserting recess of the sidewall; and
the inserting tab of each one of the supporting arms is mounted in the inserting recess of one of the sidewalls of the sole from the top edge of a corresponding sidewall of the sole and having
a top end; and
at least one connecting hole formed through the inserting tab and aligning with the at least one through hole of the corresponding sidewall of the sole.

11. The rehabilitation footwear as claimed in claim 2, wherein
each one of the sidewalls of the sole has
an inner side facing the inner side of the other one of the sidewalls of the sole;
a top edge;
an inserting recess formed in the inner side of the sidewall of the sole and is formed through the top edge of the sidewall near the rear end of the sole; and
at least one through hole formed through the sidewall of the sole and communicating with the inserting recess of the sidewall; and
the inserting tab of each one of the supporting arms is mounted in the inserting recess of one of the sidewalls of the sole from the top edge of a corresponding sidewall of the sole and having
a top end; and
at least one connecting hole formed through the inserting tab and aligning with the at least one through hole of the corresponding sidewall of the sole.

12. The rehabilitation footwear as claimed in claim 3, wherein
each one of the sidewalls of the sole has
an inner side facing the inner side of the other one of the sidewalls of the sole;
a top edge;
an inserting recess formed in the inner side of the sidewall of the sole and is formed through the top edge of the sidewall near the rear end of the sole; and
at least one through hole formed through the sidewall of the sole and communicating with the inserting recess of the sidewall; and
the inserting tab of each one of the supporting arms is mounted in the inserting recess of one of the sidewalls of the sole from the top edge of a corresponding sidewall of the sole and having
a top end; and
at least one connecting hole formed through the inserting tab and aligning with the at least one through hole of the corresponding sidewall of the sole.

13. The rehabilitation footwear as claimed in claim 1, wherein the sole has
a pad mounted on the top of the sole between the front end, the rear end and the sidewalls of the sole; and
a shock-absorbing layer mounted on the bottom of the sole.

14. The rehabilitation footwear as claimed in claim 2, wherein the sole has
a pad mounted on the top of the sole between the front end, the rear end and the sidewalls of the sole; and
a shock-absorbing layer mounted on the bottom of the sole.

15. The rehabilitation footwear as claimed in claim 3, wherein the sole has
a pad mounted on the top of the sole between the front end, the rear end and the sidewalls of the sole; and
a shock-absorbing layer mounted on the bottom of the sole.

16. The rehabilitation footwear as claimed in claim 4, wherein the sole has
a pad mounted on the top of the sole between the front end, the rear end and the sidewalls of the sole; and
a shock-absorbing layer mounted on the bottom of the sole.

17. The rehabilitation footwear as claimed in claim 5, wherein the sole has
a pad mounted on the top of the sole between the front end, the rear end and the sidewalls of the sole; and
a shock-absorbing layer mounted on the bottom of the sole.
